(19) United States
(12) Patent Application Publication
(10) Pub. No.: US 2017/0348203 A1
(43) Pub. Date: Dec. 7, 2017

(54) STABLE COSMETIC CLEANSING COMPOSITIONS CONTAINING COLD CREAM

(71) Applicant: Henkel AG & Co. KGaA, Duesseldorf (DE)

(72) Inventors: Heike Schelges, Willich (DE); Barbara Heide, Krefeld (DE); Clemens Riecke, Remscheid (DE); Martina Kampmann, Korschenbroich (DE); Elvira Scholz, Duesseldorf (DE)

(73) Assignee: Henkel AG & Co. KGaA, Duesseldorf (DE)

(21) Appl. No.: 15/609,019

(22) Filed: May 31, 2017

(30) Foreign Application Priority Data
Jun. 7, 2016 (DE) 102016209964.7

(51) Int. Cl. A61K 8/06; C07C 31/18; A61K 8/92; A61K 8/73; C11C 1/02; A61K 36/28; A61K 8/49

(52) U.S. Cl. CPC A61K 8/06; C11C 1/02; C07C 31/18; A61K 36/28; A61K 8/925; A61K 8/73; A61K 8/4946

(57) ABSTRACT

Cosmetic cleansing compositions and methods employing the cleansing compositions are provided herein. In an embodiment, a cosmetic cleansing composition includes:
a) at least one anionic surfactant and/or a fatty acid soap,
b) at least one animal wax and/or a derivative of an animal wax,
c) at least one vegetable oil,
d) at least one C₂-C₆ polyol,
e) at least one natural thickening agent,
f) at least one chamomile extract, and
g) allantoin.

### CROSS-REFERENCE TO RELATED APPLICATION

This application claims priority to German Application No. 102016209964.7, filed Jun. 7, 2016, which is hereby incorporated in its entirety by reference.

### TECHNICAL FIELD

The invention pertains to the field of cosmetics and relates to cleansing compositions with good skin care properties which are based on a specific mixture of substances comprising anionic surfactants and/or fatty acid soaps, animal waxes, vegetable oils, polyols, natural thickening agents, chamomile extract and allantoin.

### BACKGROUND

Cosmetic cleansing compositions usually contain surfactants. Because of their excellent foaming properties, anionic surfactants are usually used as the primary surfactants, however when used in large quantities, they may suffer from the disadvantage that they soften the skin and remove lipids from the outer layers of the skin. In (sensitive) skin, particularly after frequent cleansing with commercial cleansing agents, this can result in dry, rough and occasionally chapped skin unless it is replenished with lipids in the form of creams, lotions and/or shower creams during or after cleansing.

Many attempts have been made in the past to produce milder, kinder cosmetic cleansing agents which dry the skin out less and make a further treatment step with care products dispensable. The poor stability of mild compositions, which often contain a large proportion of conditioning components and/or a small proportion of (anionic) surfactants, still remains a problem, though.

What are known as cold creams have been known for a long time and have been produced for many centuries and are essentially formed from a mixture of plant or animal waxes, oils and water. However, the products were extremely unstable and rapidly became rancid. Over time, ways of stabilizing cold creams were discovered; the most effective which was used was a mixture of mineral oil and borax. However, all of the products suffer from the disadvantage that their consistency is too heavy and leave behind an extremely unpleasant film of grease on the treated skin.

In application EP 929 290 A2. the use of borax was dispensed with and instead, a combination of water, polyols, polyisobutenes and silicones was incorporated into a cold cream. In this manner, lighter, clear products could be developed which were highly suitable for cleansing in the sense of removing make-up.

Incorporating (even modern) cold creams into conventional cosmetic cleansing compositions containing surfactants, however, was still not possible, because the care components of the cold cream—waxes and oils—could not be stabilized in them in a satisfactory manner. Furthermore, the demand for natural, mild, caring cleansing products is increasing, whereby the use of borax, mineral oils and/or synthetic oils should be avoided as far as possible.

### BRIEF SUMMARY

Cosmetic cleansing compositions and methods employing the cleansing compositions are provided herein. In an embodiment, a cosmetic cleansing composition includes: at least one anionic surfactant and/or a fatty acid soap,

a) at least one animal wax and/or a derivative of an animal wax,

b) at least one vegetable oil,

c) at least one C₂-C₆ polyol,

d) at least one natural thickening agent,

e) at least one chamomile extract, and

f) allantoin.

In another embodiment, a method includes applying a cosmetic cleansing substance to skin, wherein the cosmetic cleansing composition comprises:

a) at least one anionic surfactant and/or a fatty acid soap,

b) at least one animal wax and/or a derivative of an animal wax,

c) at least one vegetable oil,

d) at least one C₂-C₆ polyol,

e) at least one natural thickening agent,

f) at least one chamomile extract, and

g) allantoin.

The method further includes rinsing the cosmetic cleansing substance from the skin.

### DETAILED DESCRIPTION

The following detailed description is merely exemplary in nature and is not intended to limit the hair treatment agents and methods for treating hair. Furthermore, there is no intention to be bound by any theory presented in the preceding background or the following detailed description.

The aim of the present disclosure was to provide mild, cosmetic cleansing compositions which care for the skin and contain components of conventional cold cream. They should have an excellent texture as well as very good stability and storage properties. Moreover, the compositions should moisturize the skin and ideally—in particular with sensitive and/or inflamed skin—have a soothing effect.

Surprisingly, it has been discovered that the aims mentioned above are accomplished to an excellent extent with the aid of cosmetic cleansing compositions in which anionic surfactants and/or soaps with specific care products (animal waxes, vegetable oils, chamomile flower extract and allantoin) in a base containing polyol and water are stabilized with natural thickening agents. The cleansing compositions are stable on storage and to heat and have very good care properties: following use, they leave the skin feeling non-greasy and smooth and in particular maintain the moisture balance of the skin during and after cleansing. Furthermore, inflamed, reddened and/or itchy skin can be soothed.

Thus, in a first aspect, the invention concerns a cosmetic cleansing composition, which contains

a) at least one anionic surfactant and/or a fatty acid soap,
b) at least one animal wax and/or a derivative of an animal wax,
c) at least one vegetable oil,
d) at least one C₂-C₆ polyol,
e) at least one natural thickening agent,
f) at least one chamomile extract, and
g) allantoin.

In a first preferred embodiment, the cleansing compositions as contemplated herein may be produced as liquid cleansing compositions such as, for example, as liquid soaps, shower gels, shower creams, shower soaps, shower balm and/or bath creams. In this case, they preferably contain the substances a) to g) in a suitable cosmetic support. In the context of the invention, preferably, this means an aqueous support which preferably contains (with respect to the total weight of the cosmetic cleansing compositions) at least about 65% by weight, more preferably at least about 70% by weight, particularly preferably at least about 75% by weight and particularly preferably at least about 80% by weight of water.

In a second preferred embodiment, the cleansing compositions as contemplated herein may be produced as solid bar soaps, syndets, combo bars and/or bath salts. In this case, in addition to the substances a) to g) (and possibly other ingredients), they preferably contain a maximum of about 15% by weight, more preferably a maximum of about 12.5% by weight and particularly preferably a maximum of about 10% by weight of water (the quantities in this regard are with respect to the total weight of the cosmetic cleansing compositions).

It has been discovered that a series of particularly mild anionic surfactants and/or soaps are suitable for the production of the compositions as contemplated herein. They provide the compositions with satisfactory foaming properties and a creamy, substantial foam. Particularly advantageously, anionic surfactants which are based on natural starting materials (such as amino acids) may also be used.

Preferred anionic surfactants a) in the context of the present disclosure may be selected from

acyl sarcosinates containing 8 to 24 C atoms in the acyl group,

acyl taurates containing 8 to 24 C atoms in the acyl group,

acyl isethionates containing 8 to 24 C atoms in the acyl group,

acyl glycinates containing 8 to 24 C atoms in the acyl group,

acyl lactylates containing 8 to 24 C atoms in the acyl group,

sulphosuccinic acid mono and/or dialkyl esters containing 8 to 24 C atoms in the alkyl group and sulphosuccinic acid monoalkyl polyoxyethylesters containing 8 to 24 C atoms in the alkyl group and 1 to 6 oxyethyl groups, and/or

alkyl sulphates and/or alkylether sulphate salts with formula R—(OCH₂—CH₂)ₙ—O—SO₃X, in which R preferably represents a linear or branched, saturated or unsaturated alkyl group containing 8 to 24 C atoms, x represents the number 0 or 1 to 12 and X represents an alkali, alkaline earth, ammonium or alkanolamine ion,

Particularly preferred anionic surfactants a) are alkali, alkaline earth and/or ammonium salts of linear or branched, saturated or unsaturated alkyl sulphates, alkylether sulphates, acyl isethionates, acyl glycinates, acyl taurates, acyl sarcosinates, acyl lactylates and/or acyl sulphosuccinates, the alkyl or acyl groups of which respectively contain 6 to 24 carbon atoms. Particularly preferred compounds are known compounds with the INCI names sodium laureth sulfate, sodium myreth sulfate, sodium trideceth sulfate, sodium lauroyl lactylate, sodium stearoyl lactylate, sodium cocoyl isethionate, sodium methyl cocoyl isethionate, sodium cocoyl glycinate, sodium methyl cocoyl glycinate, sodium cocoyl taurate, sodium methyl cocoyl taurate, sodium myristoyl sarcosinate, sodium cocoyl sarcosinate, sodium tallowate, sodium palmate and/or sodium lardate, and mixtures thereof.

The proportion by weight of the at least one anionic surfactant a) with respect to the total weight of the cosmetic cleansing compositions of the first preferred embodiment is preferably from about 0.10% to about 20.00% by weight, particularly preferably from about 0.50% to about 15.00% by weight and in particular from about 1.00% to about 10.00% by weight.

The term “fatty acid soaps” usually describes the sodium, potassium or ammonium salts of fatty acids. The term “fatty acids” should be understood to mean linear and/or branched, saturated and/or unsaturated carboxylic acids containing 6 to 30 C atoms, for example capronic acid, caprylic acid, 2-ethylhexanoic acid, capric acid, lauric, myristic, palmitic, stearic, arachidic and behenic acid, erucic acid, isostearic acid, isotridecanoic acid, and also elaidic acid, petroselenic acid, eleostearic acid, arachidic acid, gadoleic acid, palmitoleic, oleic, linoleic, linolenic, and arachidonic acid.

Synthetic mixtures of fatty acids which can be obtained from plant and animal fats and oils are preferred, for example those from tallow fatty acids, coconut fatty acids, olive oil fatty acids, palm oil fatty acids, lard fatty acids and/or palm kernel oil fatty acids. The sodium salts of the following fatty acids are particularly preferred for use in the compositions as contemplated herein: tallow fatty acid (INCI name: sodium tallowate), coconut fatty acid (INCI name: sodium cocoate), olive oil fatty acids (INCI name: sodium olivate), palm oil fatty acids (INCI name: sodium palmate), lard fatty acids (INCI name: sodium lardate) and/or palm kernel oil fatty acids (INCI name: sodium palm kernelate).

The total quantity of fatty acid soaps in the cosmetic cleansing compositions of the second preferred embodiment is preferably from about 20.0% to about 80.0% by weight, particularly preferably from about 30.0% to about 75.0% by weight and in particular from about 40.0% to about 70.0% by weight, wherein the quantities given are with respect to the total weight of the cosmetic compositions.

A third preferred embodiment of the present disclosure is provided wherein

the at least one anionic surfactant is selected from linear or branched, saturated or unsaturated alkyl sulphates, alkylether sulphates, acyl isethionates, acyl glycinates, acyl taurates, acyl sarcosinates, acyl lactylates and/or acyl sulphosuccinates, the alkyl or acyl groups of which respectively contain 6 to 24 carbon atoms, and

the at least one fatty acid soap is selected from sodium salts of tallow fatty acid (INCI name: sodium tallowate), coconut fatty acid (INCI name: sodium cocoate), olive oil fatty acids (INCI name: sodium olivate), palm oil fatty acids (INCI name: sodium palmate), lard fatty acids (INCI name: sodium lardate), and/or palm kernel oil fatty acids (INCI name: sodium palm kernelate).

In this embodiment, particularly preferred cosmetic cleansing compositions are those in which the at least one anionic surfactant and/or the fatty acid soap a) is selected from known compounds with the INCI names sodium laureth sulfate, sodium myreth sulfate, sodium trideceth sulfate, sodium lauroyl lactylate, sodium stearoyl lactylate, sodium cocoyl isethionate, sodium methyl cocoyl isethionate, sodium cocoyl glycinate, sodium methyl cocoyl glycinate, sodium cocoyl taurate, sodium methyl cocoyl taurate, sodium myristoyl sarcosinate, sodium cocoyl sarcosinate, sodium tallowate, sodium palmate and/or sodium lardate, as well as their mixtures.

A fourth preferred embodiment is provided wherein

the at least one anionic surfactant a), with respect to the total weight of the composition, is present in quantities of from about 0.10% to about 20.0% by weight, preferably in quantities of from about 0.50% to about 15.0% by weight and in particular in quantities of from about 1.00% to about 10.0% by weight, and/or

the fatty acid soap a), with respect to the total weight of the composition, is present in quantities of from about 20.0% to about 80.0% by weight, preferably in quantities of from about 30.0% to about 75.0% by weight and in particular in quantities of from about 40.0% to about 70.0% by weight.

Suitable components b) in the context of the present disclosure may preferably be selected from beeswax (cera alba) or other insect waxes, sperm whale oil, lanolin, rump fat and/or derivatives of said waxes. Suitable wax derivatives are preferably wax derivatives modified with polyalkylene glycol and/or polyglycerin because, in addition to their care properties, they can support the emulsification of vegetable oils c) in the cleansing compositions as contemplated herein. In order to obtain optimized stability of the compositions as contemplated herein, the at least one animal wax and/or wax derivative b) should preferably be solid at about 25° C. and have a melting range of from about 45° C. -85° C., more preferably from about 50° C. -80° C. and in particular from about 55° C. to about 75° C. In the context of the present disclosure, particularly preferred beeswax is that modified with polyalkylene glycol and/or polyglycerin; those known beeswax derivatives with the INCI names PEG-(2-25) beeswax and/or polyglyceryl-(2-10) beeswax are particularly preferred. Particularly preferably, the cleansing compositions as contemplated herein contain, as component b), beeswax or a known beeswax derivative with the INCI names PEG-3 beeswax, PEG-8 beeswax and/or polyglyceryl-3 beeswax—preferably beeswax (cera alba).

The proportion by weight of the at least one animal wax and/or wax derivative b) with respect to the total weight of the compositions as contemplated herein is preferably from about 0.01% to about 10.0% by weight, particularly preferably from about 0.05% to about 5.0% by weight and in particular from about 0.10% to about 3.0% by weight.

In a fifth preferred embodiment, preferred cleansing compositions as contemplated herein are those which contain beeswax or another insect wax, sperm whale oil, lanolin, rump fat and/or a derivative of said waxes as the animal wax and/or wax derivative.

In this embodiment, particularly preferred cleansing compositions as contemplated herein are those which contain beeswax and/or at least one known beeswax derivative with the INCI names PEG-(2-25) beeswax and/or polyglyceryl-(2-10) beeswax as the animal wax and/or wax derivative b).

This embodiment particularly preferably encompasses cleansing compositions as contemplated herein which contain, as the animal wax and/or wax derivative b), beeswax or a known beeswax derivative with the INCI names PEG-3 beeswax, PEG-8 beeswax and/or polyglyceryl-3 beeswax—preferably beeswax.

Preferred vegetable oils c) in the context of the present disclosure are the following oils: coconut oil, (sweet) almond oil, walnut oil, peach kernel oil, apricot kernel oil, avocado oil, tea tree oil, soya oil, cotton seed oil, sesame oil, sunflower oil, tsubaki oil, evening primrose oil, rice bran oil, palm oil, palm kernel oil, linseed oil, mango kernel oil, cranberry oil, sea buckthorn oil, cuckoo flower oil, thistle oil, macadamia nut oil, grapeseed oil, amaranth seed oil, argan oil, bamboo oil, olive oil, wheatgerm oil, pumpkin seed oil, mallow oil, hazelnut oil, castor oil, safflower oil, corn oil, olive oil, rapeseed oil, canola oil, sasanqua oil, jojoba oil, rambutan oil, cocoa butter and/or shea butter. Soya oil, cotton seed oil, sunflower oil, palm oil, palm kernel oil, linseed oil, almond oil, castor oil, corn oil, olive oil, rapeseed oil, sesame oil, soya oil, thistle oil, wheatgerm oil, peach kernel oil, cranberry oil, sea buckthorn oil and/or coconut oil are particularly preferred; and sunflower oil is especially preferred.

Optimal care properties and excellent stabilities for the cleansing compositions as contemplated herein may be obtained when a combination of the following is used as the care product mixture:

beeswax (cera alba), PEG-3 beeswax, PEG-8 beeswax and/or polyglyceryl-3 beeswax and

soya oil, cotton seed oil, sunflower oil, palm oil, palm kernel oil, linseed oil, almond oil, castor oil, corn oil, olive oil, rapeseed oil, sesame oil, soya oil, thistle oil, wheatgerm oil, peach kernel oil, cranberry oil, sea buckthorn oil and/or coconut oil;

in particular beeswax (cera alba) and sunflower oil.

The proportion by weight of the at least one vegetable oil c) with respect to the total weight of the compositions as contemplated herein is preferably from about 0.01% to about 20.0% by weight, particularly preferably from about 0.05% to about 15.0% by weight and in particular from about 0.10% to about 10.0% by weight.

In a sixth preferred embodiment, preferred cleansing compositions as contemplated herein contain soya oil, cotton seed oil, sunflower oil, palm oil, palm kernel oil, linseed oil, almond oil, castor oil, corn oil, olive oil, rapeseed oil, sesame oil, soya oil, thistle oil, wheatgerm oil, peach kernel oil, cranberry oil, sea buckthorn oil and/or coconut oil as the vegetable oil c), in particular sunflower oil.

In the context of the present disclosure, the term “suitable C₂-C₆ polyols d)” should be understood to mean polyols which contain 2 to 6 carbon atoms, preferably 2 to 4 carbon atoms, and 2 to 6 hydroxyl groups, preferably 2 or 3 hydroxyl groups in their molecule. Examples of preferred C₂-C₆ polyols d) are: ethylene glycol, 1,2-propylene glycol, 1,3-propylene glycol, dipropylene glycol, 1,3-butylene glycol, 1,4-butylene glycol, isoprene glycol, 1,2-pentanediol, 1,3-pentanediol, 1,2-hexanediol, 1,6-hexanediol, glycerin, diglycerin, sorbitol or mixtures of said polyols. Water-soluble alcohols are particularly preferred. 1,2-propylene glycol, glycerin, 1,3-butylene glycol, 1,6-hexanediol, diglycerin and/or dipropylene glycol are particularly preferred; glycerin is particularly preferred.

The proportion by weight of the at least one C₂-C₆ polyols d) with respect to the total weight of the compositions as contemplated herein is preferably from about 1.0% to about 25.0% by weight, particularly preferably from about 2.0% to about 20.0% by weight and in particular from about 3.0% to about 15.0% by weight.

In a seventh preferred embodiment, preferred cleansing compositions as contemplated herein are those which contain 1,2-propylene glycol, glycerin, 1,3-butylene glycol, 1,6-hexanediol, diglycerin and/or dipropylene glycol as the C₂-C₆ polyol d), preferably glycerin.

In the context of the present disclosure, the term “natural thickening agents” should preferably be understood to mean natural hydrocolloids. It has been discovered that natural hydrocolloids are particularly suitable for stabilizing the care products b) and c) in a base formed from water, polyols d) and surface-active substances a) over a long period of time. Furthermore, using natural hydrocolloids in the compositions as contemplated herein results in the production of cleansing agents with a very pleasant texture.

Examples of preferred natural thickening agents e) are:
polysaccharides such as glucans, modified and unmodified starches, amylose, amylopectins, dextrans, cellulose and their derivatives (methylcellulose, hydroxyalkylcellulose, ethylhydroxyethylcellulose and carboxymethylcellulose), mannans, xylans, lignins, chitin, chitosans, pectins, alginic acids and alginates, arabinogalactans, carrageenans and carrageenates, agar, gums (arabicum, karaya), carob bean gum, galactomannans such as guar gum and its non-ionic derivatives (hydroxypropyl guar), xanthan gum, scleroglucan and mixtures thereof.

Particularly good results as regards stability and texture of the compositions as contemplated herein may be obtained when at least one carrageenan and/or carrageenate is used as the natural thickening agent e).

The proportion by weight of the at least one natural thickening agent e) with respect to the total weight of the compositions as contemplated herein is preferably from about 0.01% to about 3.00% by weight, particularly preferably from about 0.10% to about 2.00% by weight and in particular from about 0.20% to about 1.50% by weight.

In an eighth preferred embodiment, preferred cleansing compositions as contemplated herein are those containing polysaccharides such as glucans, modified and unmodified starches, amylose, amylopectins, dextran, cellulose and their derivatives (methylcellulose, hydroxyalkylcellulose, ethylhydroxyethylcellulose and carboxymethylcellulose), mannans, xylans, lignins, chitin, chitosans, pectins, alginic acids and alginates, arabinogalactans, carrageenans and carrageenates, agar, gums (arabicum, karaya), carob bean gum, galactomannans such as guar gum and its non-ionic derivatives (hydroxypropyl guar), xanthan gum, scleroglucan and mixtures thereof as the at least one natural thickening agent e). Particularly preferred cleansing compositions as contemplated herein in this embodiment contain carrageenans and/or carrageenates as the natural thickening agent e).

The cleansing compositions as contemplated herein contain, as the further essential substance, at least one chamomile extract f), which is preferably used in a proportion by weight of from about 0.001% to about 1.0% by weight, particularly preferably from about 0.003% to about 0.75% by weight and in particular from about 0.005% to about 0.5% by weight with respect to the total weight of the compositions as contemplated herein. The chamomile extract may be obtained using known production processes—for example by milling—from any parts of plants or from individual parts of the chamomile (such as leaves, flowers, roots), with optional subsequent drying and/or extraction. The extraction is usually carried out at raised temperatures in an extractor (for example a Soxhlet). In this regard, suitable solvents are used, depending on whether the oil- or water-soluble components of the plant (parts) are required. The extraction agent for the production of the cited plant extracts may, for example, be water, alcohols as well as their mixtures. Examples of alcohols which may be used are lower alcohols such as ethanol and isopropanol, but also in particular polyalcohols such as ethylene glycol, propylene glycol, butylene glycol, glycerin and/or sorbitol, both as the sole extraction agent and also as a mixture with water. In this regard, plant extracts based on water/alcohol(s) in a ratio of from about 1:10 to about 10:1 have been shown to be particularly suitable, for example. Extracts from chamomile flowers have been shown to be particularly suitable for the cleansing agents as contemplated herein because of their excellent skin soothing effects. These may be oil-based, aqueous or hydroalcoholic. Appropriate extracts are commercially available from various suppliers as a mixture with the respective extraction agent, for example from the firms Cosmetochem, Symrise or Croda.

In a ninth preferred embodiment, preferred cleansing compositions as contemplated herein are those which contain an aqueous, hydroalcoholic or oil-based chamomile flower extract as the chamomile extract f) in a proportion by weight of from about 0.001% to about 1.0% by weight, preferably from about 0.003% to about 0.75% by weight and in particular from about 0.005% to about 0.5% by weight with respect to the total weight of the cleansing composition.

In a tenth preferred embodiment, preferred cleansing compositions as contemplated herein are those which contain allantoin g) in a proportion by weight of from about 0.001% to about 1.0% by weight, preferably in quantities of from about 0.005% to about 0.75% by weight and in particular in quantities of from about 0.010% to about 0.5% by weight with respect to the total weight of the cleansing compositions.

It has been discovered that the combination of substances b), c), f) and g) forms a highly effective skin care complex in the cleansing compositions as contemplated herein which not only regenerates dry, sensitive and/or inflamed skin and provides it with more moisture and smoothness, but also alleviates or even removes feelings of tightness and/or itchiness.

A care complex which (with respect to the total weight of the cosmetic cleansing composition as contemplated herein) contains the following is particularly preferred:

From about 0.10% to about 3.0% by weight of beeswax (cera alba),

From about 0.10% to about 10.0% by weight of sunflower oil,

From about 0.005% to about 0.5% by weight of chamomile flower extract, and

From about 0.01% to about 0.5% by weight of allantoin.

The stability and texture of liquid cleansing compositions in particular as contemplated herein can be still further improved by using a higher fatty alcohol. The term “higher fatty alcohols” as used in the context of the present disclosure should be understood to mean linear or branched, saturated or unsaturated alcohols containing 12 to 32. preferably 16 to 26 and in particular 20 to 24 carbon atoms. Arachidyl alcohol, behenyl alcohol and/or lignoceryl alcohol are particularly preferred; behenyl alcohol is more particularly preferred. The higher fatty alcohol(s) may preferably be used in the cleansing compositions as contemplated herein in a proportion by weight of from about 1.0% to about 10.0% by weight, particularly preferably from about 2.0% to about 8.0% by weight and in particular from about 3.0% to about 6.0% by weight with respect to the total weight of the cleansing composition.

In a further preferred embodiment, the cleansing compositions as contemplated herein furthermore contain a higher alcohol—preferably behenyl alcohol—in a proportion by weight of from about 1.0% to about 10.0% by weight, particularly preferably from about 2.0% to about 8.0% by weight and in particular from about 3.0% to about 6.0% by weight with respect to the total weight of the cleansing composition.

In order to further enhance the mildness and/or foaming properties of the cleansing compositions as contemplated herein, in a further preferred embodiment, it may be advantageous for the compositions—with respect to their weight—to additionally contain from about 0.10% to about 15.00% by weight, particularly preferably from about 0.50% to about 10.00% by weight and in particular from about 0.75% to about 7.50% by weight of at least one amphoteric, zwitterionic and/or nonionic surfactant.

More particularly preferred amphoteric and/or zwitterionic surfactants are known amphoteric/zwitterionic surfactants which are commercially available from a number of suppliers and which have the INCI names sodium cocoamphoacetate, disodium cocoamphodiacetate, sodium lauroamphoacetate, sodium lauroamphodiacetate, sodium cocoamphopropionate, disodium cocoamphodipropionate, cocoamidopropyl hydroxysultaine, coco sultaine, lauryl sultaine, coco betaine, lauryl betaine cocamidopropylbetain and/or lauramidopropylbetain. Cocamidopropylbetain and/or cocoampho(di)acetate are particularly preferred.

Particularly preferred nonionic surfactants are:

fatty acid alkanolamides, preferably C₈-C₂₄ carboxylic acid mono- and/or di-(C₂-C₄)-alkanolamides and in particular those known compounds with the INCI names cocamide MEA and/or cocamide MIPA,

alkylpolyglucosides, preferably those known compounds with the INCI names coco glucoside, lauryl glucoside and/or decyl glucoside,

C₈-C₃₀ fatty acid mono- and diesters of addition products from 1 to 30 mols of ethylene oxide and glycerin, in particular the known compound with the INCI name PEG-7 glyceryl cocoate,

C₈-C₃₀ fatty acid mono- and diesters of glycerin, in particular the known compound with the INCI name glyceryl caprylate/caprate,

glucamides, in particular those known compounds with the INCI names cocoyl methyl glucamide or lauroyl/myristoyl methyl glucamide.

Cocamide MEA and/or PEG-7 glyceryl cocoate are particularly preferred because of their foam-stabilizing and/or relubrication properties.

In addition to the essential and optional components cited above, the cosmetic cleansing compositions as contemplated herein may contain at least one further conditioning substance to further enhance the care properties of the agent, preferably a skin conditioning substance from the group formed by cationic polymers and/or vitamins.

Examples of suitable cationic polymers are as follows:

quaternized cellulose derivatives, such as those which are commercially available under the trade names Celquat® and polymer JR®,

hydrophobic modified cellulose derivatives, for example those cationic polymers marketed with the trade name SoftCat®,

cationic alkylpolyglycosides,

honey which has been rendered cationic, for example the commercially available product Honeyquat® 50.

cationic guar derivatives, in particular such as those marketed under the trade names Cosmedia Guar N-Hance® and Jaguar®,

polymeric dimethyldiallylammonium salts and their copolymers with esters and amides of acrylic acid and methacrylic acid. Examples of such cationic polymers are those which are commercially available under the trade names Merquat®100

(poly(dimethyldiallylammonium chloride)) and Merquat®550 (dimethyl diallyl ammonium chloride—acrylamide copolymer),

copolymers of vinylpyrrolidone with quaternized derivatives of dialkylaminoalkyl acrylates and methacrylates such as, for example, with diethylsulphate quaternized vinylpyrrolidone-dimethylaminoethylmethacrylate copolymers. Such compounds are commercially available under the descriptions Gafquat®734 and Gafquat®755,

vinylpyrrolidone-vinylimidazoliummethochloride copolymers, such as those which are sold under the trade names Luviquat® FC 370. FC 550. FC 905 and HM 552.

quaternized polyvinyl alcohol, as well as those polymers which are known by the names:

polyquaternium 2, polyquaternium 17, polyquaternium 18, polyquaternium 24, polyquaternium 27, polyquaternium 32, polyquaternium 37, polyquaternium 74 and polyquaternium 89.

Preferred cationic polysaccharide polymers are those such as quaternized cellulose polymers, hydrophobic modified cationic cellulose derivatives and/or cationic guar derivatives, as well as polymeric dimethyldiallyl ammonium salts and their copolymers with esters and amides of acrylic acid and methacrylic acid. Particularly preferred compounds are those known cationic polymers with the INCI names guar hydroxypropyltrimonium chloride, polyquaternium 10, polyquaternium 37, polyquaternium 67, polyquaternium 72, polyquaternium 6 and/or polyquaternium 7; polyquaternium 7 is especially preferred. The cosmetic cleansing compositions as contemplated herein contain cationic polymers—preferably polyquaternium 7—in a proportion by weight of from about 0.01% to about 3.00% by weight, particularly preferably from about 0.10% to about 2.00% by weight and in particular from about 0.20% to about 1.50% by weight with respect to the total weight of the cleansing compositions.

The term “suitable vitamins” should preferably be understood to mean the following vitamins, provitamins and vitamin precursors as well as their derivatives:

Vitamin A: the following substances belong to the group designated as vitamin A: retinol (vitamin A₁) as well as 3,4-didehydroretinol (vitamin A₂). β-carotin is the provitamin of retinol. Examples of vitamin A components are vitamin A acids and their esters, vitamin A aldehyde and vitamin A alcohol, as well as their esters such as the palmitate and acetate.

Vitamin B: the vitamin B group of vitamin B complex includes, inter alia:
vitamin B₁ (thiamine)
vitamin B₂ (riboflavin)
vitamin B₃. This designation often includes the compounds nicotinic acid and nicotinic acid amide (niacinamide).
vitamin B₅ (pantothenic acid and panthenol). In the context of this group, panthenol is preferably used. Derivatives of panthenol which may in particular be used are the esters and ethers of panthenol, pantolactone as well as cationic derivatized panthenols. Examples of individual representatives are panthenol triacetate, panthenol monoethyl ether and its monoacetate, as well as cationic panthenol derivatives.
vitamin B₆ (pyridoxine as well as pyridoxamine and pyridoxal).

Vitamin C (ascorbic acid): use in the form of the palmitic acid ester, the glucosides or phosphates may be advantageous. Use in combination with tocopherols may also be advantageous.

Vitamin E (tocopherols, in particular α-tocopherol).

Vitamin F: the term “vitamin F” usually means essential fatty acids, in particular linoleic acid, linolenic acid and arachidic acid.

Vitamin H: the compound (3aS,4S,6aR)-2-oxohexahydrothienol[3,4-d]-imidazol-4-valeric acid is described as vitamin H; it is now usually known by the trivial name of biotin.

Particularly preferred vitamins, provitamins and vitamin precursors are those from the groups A, B, E and H. Nicotinic acid amide, biotin, pantolactone and/or panthenol are particularly preferred; nicotinic acid amide is especially preferred.

The proportion by weight of the vitamin(s), vitamin derivative(s), and/or the vitamin precursor(s) with respect to the total weight of the cosmetic cleansing compositions as contemplated herein is preferably from about 0.005% to about 2.00% by weight, more preferably from about 0.006% to about 1.50% by weight, particularly preferably from about 0.008% to about 1.00% by weight and in particular from about 0.01% to about 0.50% by weight.

Further examples of substances, excipients and additives which may be used in the cosmetic cleansing compositions as contemplated herein are as follows:

UV filters,

structuring agents such as maleic acid and lactic acid,

colorants in order to colour the medium,

substances for adjusting the pH, for example α- and β-hydroxycarboxylic acids such as citric acid, lactic acid, malic acid, glycolic acid,

complexing agents such as EDTA, NTA, β-alanine diacetic acid and phosphonic acids,

ceramides. The term “ceramide” should be understood to mean N-acylsphingosine (fatty acid amide of sphingosines) or synthetic analogues of such lipids (what are known as pseudo-ceramides),

propellants such as propane-butane mixtures, N₂O, dimethylether, CO₂ and air,

antioxidants,

pearlizing agents such as EGDS or PEG-3 distearate,

opacifiers such as the known compounds with the INCI name styrene/acrylates copolymer,

fragrance components,

viscosity regulators such as sodium chloride (NaCl).

In a second aspect, the present disclosure concerns the cosmetic use of a combination comprising:

a) at least one anionic surfactant and/or a fatty acid soap,
b) at least one animal wax and/or a derivative of an animal wax,
c) at least one vegetable oil,
d) at least one C₂-C₆ polyol,
e) at least one natural thickening agent,
f) at least one chamomile extract, and
g) allantoin
in rinse-off skin cleansing substances, for the purposes of

improving care properties;

improving the skin soothing effect;

improving moisturizing properties;

improving stability; and

improving the texture of the skin care substance.

The above discussions relating to the agents as contemplated herein apply mutatis mutandis to the preferred embodiments of the uses as contemplated herein.

### Examples

I. The following liquid cosmetic cleansing compositions as contemplated herein were produced (the quantities refer to the % by weight):

| | 1 | 2 | 3 | 4 | 5 | 6 |
|---|---|---|---|---|---|---|
| Sodium laureth sulfate | 3.00 | 1.25 | 1.25 | 1.25 | | |
| Sodium trideceth sulfate | | | | | 8.00 | 8.00 |
| Sodium stearoyl lactylate | 0.50 | 0.50 | 0.50 | 0.50 | | |
| Beeswax (cera alba) | 0.15 | 0.15 | 0.15 | 0.15 | 0.25 | |
| PEG-8 beeswax | | | | | | 0.25 |
| Olive oil | 1.00 | | | | | |
| Sunflower oil | | 1.00 | 1.00 | 1.00 | 15.0 | 15.0 |
| Glycerin | 5.00 | 5.00 | 5.00 | 5.00 | 7.00 | 7.00 |
| carrageenan | 1.15 | 1.15 | 1.15 | 1.15 | | |
| Xanthan Gum | | | | | 0.80 | 0.80 |
| Chamomilla recutita matricaria flower | 0.10 | 0.10 | 0.20 | 0.10 | 0.10 | 0.10 |
| extract | | | | | | |
| Allantoin | 0.10 | 0.10 | 0.05 | 0.01 | 0.01 | 0.01 |
| Behenyl alcohol | 3.00 | 2.00 | 3.00 | 2.00 | | |
| Cocamidopropylbetain | 5.70 | 5.70 | 5.70 | 5.70 | | |
| Sodium lauroamphoacetate | | | | | 2.50 | 2.50 |
| Cocamide MEA | | | | | 1.75 | 1.75 |
| Fragrance | 1.00 | 1.00 | 1.00 | 1.00 | 0.80 | 0.80 |
| Polyquaternium 7 | | | | | 0.25 | 0.25 |
| Niacinamide | 0.10 | | | | | |
| Tocopherol | | | | | 0.03 | 0.03 |
| Preservative | q.s. | q.s. | q.s. | q.s. | q.s. | q.s. |
| pH-adjusting agent (to pH 4.5-5.5) | q.s. | q.s. | q.s. | q.s. | q.s. | q.s. |
| Water | ad | ad | ad | ad | ad | ad |
| | 100 | 100 | 100 | 100 | 100 | 100 |

1, 3: Shower balsam;
2: Bath cream;
4: Liquid soap;
5, 6: Shower cream

Compared with comparative formulations (in which one or more of the substances a) to g) were missing), the cosmetic cleansing compositions as contemplated herein of Examples 1-6 exhibited an improved stability and texture. They had a long-lasting action on rough and/or flaky skin and provided it with a smooth, soft skin feel which was still discernible long after rinsing off the compositions.

II. The following solid cosmetic cleansing compositions as contemplated herein were produced (the quantities refer to the % by weight):

| | Solid soap |
|---|---|
| Sodium tallowate | 40.0 |
| Sodium cocoate | 24.0 |
| Beeswax (cera alba) | 0.15 |
| Sunflower oil | 1.00 |
| Glycerin | 4.00 |
| carrageenan | 1.00 |
| Chamomilla recutita matricaria flower extract | 0.01 |
| Allantoin | 0.01 |
| Talc | 10.0 |
| Coconut Acid | 4.00 |
| Sodium myreth sulfate | 2.00 |
| Decyl glucoside | 0.50 |
| Fragrance | 1.00 |
| Preservative | q.s. |
| Water | ad 100 |

While at least one exemplary embodiment has been presented in the foregoing detailed description, it should be appreciated that a vast number of variations exists. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic cleansing composition, containing comprising:
a) at least one anionic surfactant and/or a fatty acid soap,
b) at least one animal wax and/or a derivative of an animal wax,
c) at least one vegetable oil,
d) at least one C₂-C₆ polyol,
e) at least one natural thickening agent,
f) at least one chamomile extract, and
g) allantoin.

2. The cosmetic cleansing composition as claimed in 1, wherein:
the at least one anionic surfactant is selected from linear or branched, saturated or unsaturated alkyl sulphates, alkylether sulphates, acyl isethionates, acyl glycinates, acyl taurates, acyl sarcosinates, acyl lactylates and/or acyl sulphosuccinates, the alkyl or acyl groups of which respectively comprise 6 to 24 carbon atoms, as well as their mixtures, and
the at least one fatty acid soap is selected from sodium salts of tallow fatty acid (INCI name: sodium tallowate), coconut fatty acid (INCI name: sodium cocoate), olive oil fatty acids (INCI name: sodium olivate), palm oil fatty acids (INCI name: sodium palmate), lard fatty acids (INCI name: sodium lardate) and/or palm kernel oil fatty acids (INCI name: sodium palm kernelate), as well as their mixtures.

3. The cosmetic cleansing composition as claimed in claim 1, wherein:
the at least one anionic surfactant a), with respect to the total weight of the composition, is present in quantities of from about 0.10% to about 20.0% by weight, and/or
the fatty acid soap a), with respect to the total weight of the composition, is present in quantities of from about 20.0% to about 80.0% by weight.

4. The cosmetic cleansing composition as claimed in claim 1, wherein
the at least one animal wax and/or its derivative b) is selected from beeswax or other insect waxes, sperm whale oil, lanolin, rump fat and/or derivatives of said waxes.

5. The cosmetic cleansing composition as claimed in claim 4, wherein the at least one animal wax and/or its derivative b) is selected from beeswax and/or from at least one known beeswax derivative with the INCI names PEG-(2-25) beeswax and/or polyglyceryl-(2-10) beeswax.

6. The cosmetic cleansing composition as claimed in claim 1, wherein
the at least one animal wax and/or its derivative b), with respect to the total weight of the composition, is present in quantities of from about 0.01% to about 10.0% by weight.

7. The cosmetic cleansing composition as claimed in claim 1, wherein
the at least one vegetable oil c) is selected from soya oil, cotton seed oil, sunflower oil, palm oil, palm kernel oil, linseed oil, almond oil, castor oil, corn oil, olive oil, rapeseed oil, sesame oil, soya oil, thistle oil, wheatgerm oil, peach kernel oil, cranberry oil, sea buckthorn oil and/or coconut oil.

8. The cosmetic cleansing composition as claimed in claim 1, wherein
the at least one vegetable oil c), with respect to the total weight of the composition, is present in quantities of from about 0.01% to about 20.0% by weight.

9. The cosmetic cleansing composition as claimed in claim 1, wherein
the at least one C₂-C₆ polyol d) is selected from 1,2-propylene glycol, glycerin, 1,3-butylene glycol, 1,6-hexanediol, diglycerin and/or dipropylene glycol.

10. The cosmetic cleansing composition as claimed in claim 1, wherein
the at least one C₂-C₆ polyol d), with respect to the total weight of the composition, is present in quantities of from about 1.0% to about 25.0% by weight.

11. The cosmetic cleansing composition as claimed in claim 1, wherein
the at least one natural thickening agent e) is selected from polysaccharides such as glucans, modified and unmodified starches, amylose, amylopectins, dextrans, celluloses and their derivatives (methylcelluloses, hydroxyalkylcelluloses, ethylhydroxyethylcelluloses and carboxymethylcelluloses), mannans, xylans, lignins, chitin, chitosans, pectins, alginic acids and alginates, arabinogalactans, carrageenans and carrageenates, agar, gums (arabicum, karaya), carob bean gum, galactomannans such as guar gum and non-ionic derivatives (hydroxypropyl guar), xanthan gum, scleroglucan, and mixtures thereof.

12. The cosmetic cleansing composition as claimed in claim 1, wherein
the at least one natural thickening agent e), with respect to the total weight of the composition, is present in quantities of from about 0.01% to about 3.00% by weight.

13. The cosmetic cleansing composition as claimed in claim 1, comprising an aqueous, hydroalcoholic or oil-based chamomile flower extract as the chamomile extract f) which, with respect to the total weight of the composition, is present in quantities of from about 0.001% to about 1.0% by weight.

14. The cosmetic cleansing composition as claimed in claim 1 comprising, with respect to the total weight of the composition, allantoin g) in quantities of from about 0.001 to about 1.0% by weight.

15. A method comprising:
applying a cosmetic cleansing composition to skin, wherein the cosmetic cleansing composition comprises:
a. at least one anionic surfactant and/or a fatty acid soap,
b. at least one animal wax and/or a derivative of an animal wax,
c. at least one vegetable oil,
d. at least one C₂-C₆ polyol,
e. at least one natural thickening agent,
f. at least one chamomile extract, and
g. allantoin; and
rinsing the cosmetic cleansing substance from the skin.

16. The cosmetic cleansing composition as claimed in 1, wherein the at least one anionic surfactant is selected from compounds with the INCI names sodium laureth sulfate, sodium myreth sulfate, sodium trideceth sulfate, sodium lauroyl lactylate, sodium stearoyl lactylate, sodium cocoyl isethionate, sodium methyl cocoyl isethionate, sodium cocoyl glycinate, sodium methyl cocoyl glycinate, sodium cocoyl taurate, sodium methyl cocoyl taurate, sodium myristoyl sarcosinate, as well as their mixtures.

17. The cosmetic cleansing composition as claimed in 1, wherein the at least one fatty acid soap is present and is selected from compounds with the INCI names sodium tallowate, sodium palmate and/or sodium lardate, as well as their mixtures.

18. The cosmetic cleansing composition as claimed in claim 3, wherein the at least one anionic surfactant a), with respect to the total weight of the composition, is present in quantities of from about 1.00% to about 10.0% by weight, and wherein the fatty acid soap a), with respect to the total weight of the composition, is present in quantities of from about 40.0% to about 70.0% by weight.

19. The cosmetic cleansing composition as claimed in claim 1, wherein:
the at least one animal wax and/or its derivative b), with respect to the total weight of the composition, is present in quantities of from about 0.10% to about 3.0% by weight,
the at least one vegetable oil c), with respect to the total weight of the composition, is present in quantities of from about 0.10% to about 10.0% by weight,
the at least one chamomile extract f), with respect to the total weight of the composition, is present in quantities of from about 0.005% to about 0.5% by weight,
the allantoin g), with respect to the total weight of the composition, is present in quantities of from about 0.010% to about 0.5% by weight.

20. The method of claim 15, wherein the cosmetic cleansing composition is applied to skin for one or more of the following purposes:
improving care properties;
improving the skin soothing effect;
improving moisturizing properties;
improving stability; and
improving the texture of the skin care substance.
